(19) United States
(12) Patent Application Publication
(10) Pub. No.: US 2026/0158226 A1
(43) Pub. Date: Jun. 11, 2026

(54) SYRINGE IDENTIFICATION

(71) Applicant: OWEN MUMFORD LTD, Woodstock, Oxfordshire (GB)

(72) Inventor: Timothy Simon EVANS, Woodstock, Oxfordshire (GB)

(21) Appl. No.: 18/707,110

(22) PCT Filed: Nov. 2, 2022
(86) PCT No.: PCT/EP2022/080582

§ 371 Date: May 2, 2024

(30) Foreign Application Priority Data
Nov. 3, 2021 (GB) 2115806.8

(51) Int. Cl. A61M 5/32; A61M 5/20

(52) U.S. Cl. CPC A61M 5/3245; A61M 5/2033; A61M 5/3257; A61M 2205/0233; A61M 2205/3592; A61M 2205/6009; A61M 2205/8206

(57) ABSTRACT

A syringe comprising a barrel and a needle at an end thereof, a syringe plunger configured to move within the barrel to cause a medicament within the barrel to be expelled from the needle; and an electrically erasable programmable read-only memory (EEPROM) configured to store information related to the syringe.

### TECHNICAL FIELD

The invention relates to syringes, for example but not limited to safety syringes, and to auto-injectors for operating syringes.

### BACKGROUND

Syringes are devices used to inject a substance such as a medicament or drug into a user. A syringe typically comprises a barrel containing a medicament, a needle and a syringe plunger. When the syringe plunger is driven into the barrel, the contents of the barrel are forced through the needle into an injection site on a user.

Safety syringes are types of syringes that additionally include some form of safety mechanism to users from the needle of the syringe after the injection has been completed. Safety syringes typically comprise a shroud or sheath for covering the needle after use of the syringe. Other exemplary safety syringes may cause the needle to retract within the barrel of the syringe. An exemplary safety syringe is provided in WO2016/026952A1.

Auto-injectors are devices for receiving and operating syringes automatically on activation by a user. Auto-injectors typically include a plunger driver that engages with a syringe plunger of a syringe inserted into the auto-injector and one or more drive springs that are arranged to couple to and apply a force to the syringe plunger. To inject a drug, the user holds the auto-injector against the injection site and triggers the auto-injector, causing the plunger driver to drive the syringe plunger forward. This firstly pushes the entire syringe through the injector housing resulting in needle insertion. The syringe body then bottoms out in the housing such that continued force on the plunger results in the plunger travelling into the drug-filled barrel of the syringe. The drug is thereby forced through the needle into the injection site.

Radio-frequency identification (RFID) tags typically comprise a radio transponder and a transceiver. When an electromagnetic pulse from a nearby RFID reader device triggers the RFID tag, the tag transmit digital information such as information about an object with which the RFID is associated.

Quick Response (QR) codes comprise a two-dimensional pattern of pixels that provide a machine-readable optical label containing information about an object with the QR code is associated. The information contained in the QR code may be extracted by capturing an image of it and converting the optical label into digital information.

RFID tags and QR codes are used to identify syringes and their contents prior to insertion into an auto-injector to ensure a user is being injected with the correct drug.

For example, a smartphone with an RFID reader and a camera may read the RFID tag and/or QR code on a selected syringe. A software application on the smartphone can then confirm to the user if the selected syringe and the dose of medicament it contains is the correct one before the user inserts the syringe into the auto-injector.

Alternatively, an RFID reader may be incorporated into an auto-injector. An exemplary auto-injector and a safety syringe is provided in WO2019/086718A1. The auto-injector of WO2019/086718A1 comprises an RFID reader that reads an RFID tag of a syringe inserted into the auto-injector to obtain information about the syringe and/or a its contents.

Electrically erasable programmable read-only memory (EEPROM) is a type of read-only memory. An EEPROM typically comprises a number of input and output contact pins. Establishing an electrical connection to the EEPROM through the contact pins allows information stored on the device to be read. Communication between an EEPROM and external devices is typically performed using one or more known communication protocols or busses such as I2C.

### SUMMARY

According to a first aspect there is provided a syringe comprising a barrel and a needle at an end thereof, a syringe plunger configured to move within the barrel to cause a medicament within the barrel to be expelled from the needle; and an electrically erasable programmable read-only memory (EEPROM) configured to store information related to the syringe.

Optionally, the syringe comprises a printed circuit board comprising one or more electrically conductive contact pads electrically connected to one or more input or output pins of the EEPROM.

Optionally, the syringe comprises: a safety plunger coupled to the syringe plunger such that an inward stroke of the safety plunger causes the syringe plunger to move within the barrel; a sheath configured to cover the needle after use of the syringe; wherein the safety plunger is configured to couple to the sheath at a first point on the inward stroke, and is configured to decouple from the syringe plunger at a second point on the inward stroke such that the safety plunger is moveable independently of the syringe plunger, and wherein further movement of the safety plunger after the first and second points on the inward stroke causes the sheath to cover the needle.

Optionally, the printed circuit board is arranged on a rearward facing surface of the safety plunger.

Optionally, the printed circuit board is arranged on a rearward facing surface of the syringe plunger.

According to a second aspect, there is provided an auto-injector for receiving and operating a syringe to facilitate the injection of a medicament into a subject, the autoinjector comprising: a plunger driver configured on activation of the auto-injector to drive a plunger of the syringe received in the auto-injector forward within the auto-injector to operate the syringe; and one or more electrically conductive contact pads configured to contact one or more electrically conductive contact pads on the syringe to establish an electrical connection between an integrated circuit of the auto-injector and an electrically erasable programmable read-only memory (EEPROM) of the syringe.

Optionally, the one or more electrically conductive contact pads are arranged on a forward facing surface of the plunger driver.

Optionally, the integrated circuit is configured to read the EEPROM to obtain information relating to the syringe stored on the EEPROM, and export the information to an external device.

Optionally, the integrated circuit of the auto-injector is configured to communicate with the EEPROM of the syringe using an I2C bus.

### BRIEF DESCRIPTION OF THE DRAWINGS

Exemplary embodiments are disclosed herein with reference to the accompanying drawings, in which:

FIG. 1a shows a view of a safety syringe before use.

FIG. 1b shows a view of the safety syringe of FIG. 1a after use.

FIG. 1c shows a view of the safety plunger of the safety syringe of FIG. 1a

FIG. 2a shows a view of an auto-injector.

FIG. 2b shows a view of the auto-injector of FIG. 2a with a safety syringe received therein.

FIG. 2c shows a view of a . . . of the auto-injector of FIG. 2a.

### DETAILED DESCRIPTION

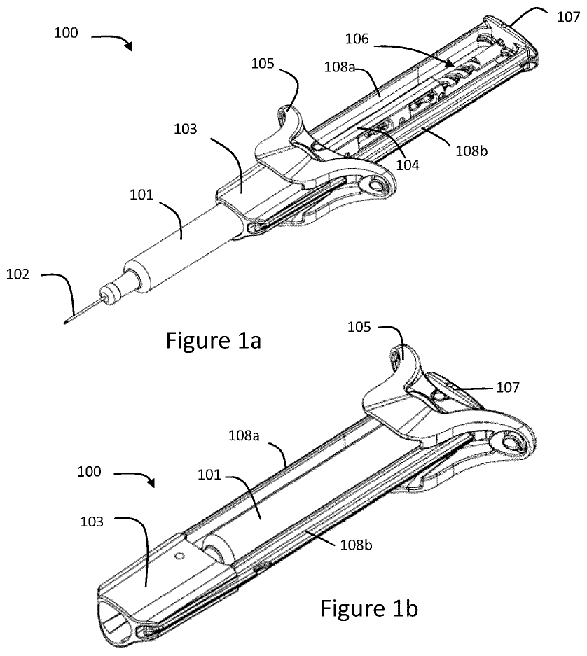

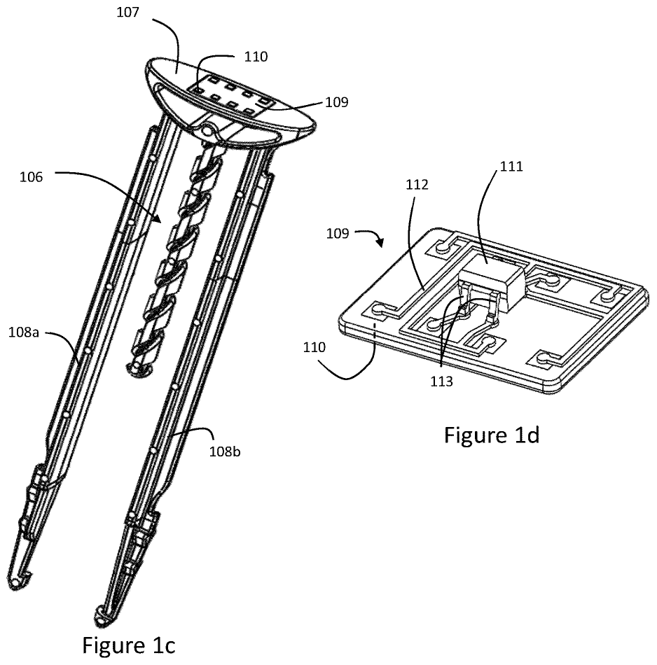

FIG. 1a shows a view of a safety syringe 100 before use. The safety syringe 100 comprises a barrel 101, a needle 102, a shroud 103, a syringe plunger 104, a handle portion 105, and a safety plunger 106 coupled to the syringe plunger 104. The safety plunger 106 further comprises a head portion 107 at a rearward end and two arms 108a, 108b coupled to the sheath 103 through apertures in the handle portion 105.

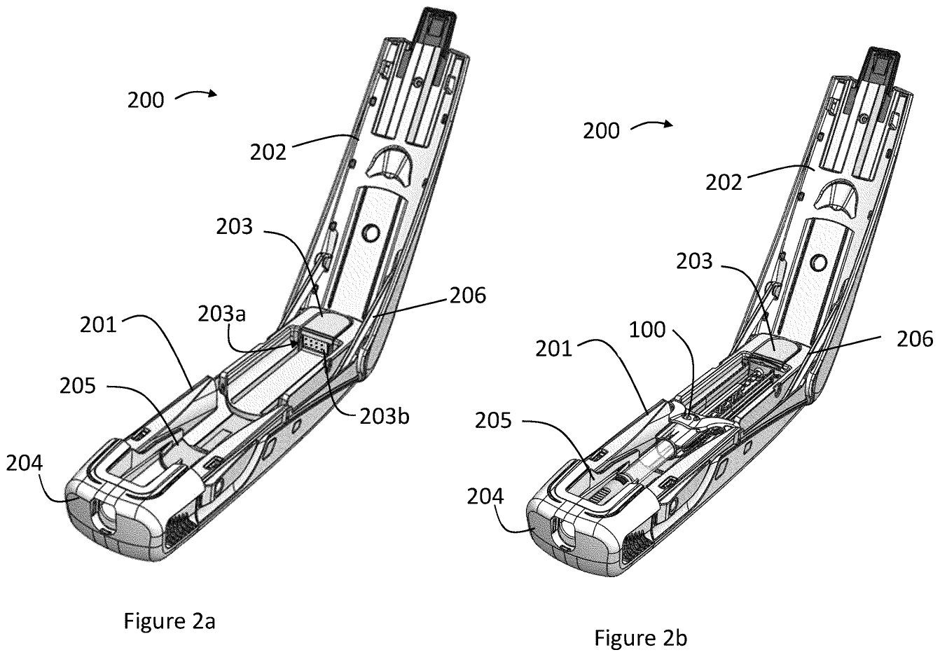

To operate the safety syringe 100, the user inserts the needle 102 into an injection site and applies a forward force to the head portion 107 of the safety plunger 106 causing it and the coupled syringe plunger 104 to move forward, driving the syringe plunger 104 into the barrel 101. The forward force may be applied by an auto-injector such as that described in FIGS. 2a-2c. As the syringe plunger 104 moves into the barrel 101, the contents of the barrel 101 are forced through the needle 102 into the injection site. Further, as the safety plunger 106 moves forward, the arms 108a, 108b of the syringe plunger cause the sheath 103 to move forward towards the injection site until it reaches user's skin.

At a predetermined point on the inward stroke, the safety plunger 106 decouples from the syringe plunger 104 such that the safety plunger 106 is moveable independently of the syringe plunger 104. While maintaining forward pressure on the head portion 107 of the safety plunger 106 to keep the shroud 103 firmly pressed on the user's skin, the handle portion 105 may be used to pull the needle 102 and barrel 101 containing the now bottomed out and decoupled syringe plunger 104 rearwards relative to the shroud 103 and safety plunger 106 which remain stationary and pressed against the user's skin. As the needle 102 exits the injection site, it is covered by the shroud 103 to protect the user from injury.

FIG. 1b shows a view of the safety syringe 100 of FIG. 1a after use. The needle 102, barrel 101 and syringe plunger 104 have moved rearwards relative to the shroud 103, handle portion 105 and safety plunger 106. In this configuration, the shroud 103 is locked out in a position covering the needle 102 to protect the user from injury and the safety syringe may thus be disposed of safely.

FIG. 1c shows a view of the safety plunger 106 of the safety syringe 106 of FIG. 1a. As described above, the safety plunger 106 comprises a head portion 107 against which a forward pressure may be applied to drive the safety plunger 106 forward, and two arms 108a, 108b to couple with a sheath 103. The safety plunger 106 further comprises an electrically erasable programmable read-only memory (EEPROM) 111mounted on a printed circuit board (PCB) 109 arranged on a rearward facing surface of the head portion 107 of the safety plunger 106. The EEPROM 111 is configured to store information related to the syringe 100 such as what medicament and what dose is contained in the barrel 101 of the syringe 100. The PCB 109 further comprises one or more electrically conductive contact pads 110 electrically connected to one or more input or output pins of the EEPROM. The EEPROM 111 may be secured to a rearward facing surface of the head portion 107 of the safety plunger 106 using glue and/or a snap fit connection in a suitably shaped slot shaped to receive the EEPROM 111 therein or on.

FIG. 1d shows a view of an opposite side of the PCB 109 of FIG. 1c. The EEPROM 111 and the opposite sides of the contact pads 110 are visible in FIG. 1d. Electrically conductive traces 112 are provided on the PCB 109 to electrically connect the contact pads 110 to input and/or output pins 113 of the EEPROM 111.

To read the information stored in the EEPROM 11, an electrical connection may established to an external device through the one or more contact pads 110 and traces 112 to the input and/or output pins 113. For example, when the safety syringe 100 is used in an auto-injector having correspondingly positioned contact pads, an electrical connection with the EEPROM 111 may be established and the information stored on the EEPROM 111 may be read by an integrated circuit in the auto-injector.

FIG. 2a shows a view of an auto-injector 200 for receiving and operating a syringe to facilitate injection of medicament into a subject. The auto-injector 200 comprises a housing 201 having an openable lid 202, a plunger driver 203 comprising a pair of drive springs (not shown), a removable cap 204. The lid 202 is configured to open to allow insertion and removal of a syringe, for example the safety syringe described in connection with FIGS. 1a-1c, in the auto-injector 200 prior to use. The plunger driver 202 is configured to couple to a plunger of the syringe inserted in the auto-injector 200, for example by engaging a rearward facing surface of a head portion of the safety plunger of the safety syringe. The removable cap 204 covers a forward end of the auto-injector 200 prior to use. The lid 202 is configured to prime the drive springs on an opening and/or closing movement thereof. This may be achieved by, for example, coupling the lid 202 to the drive springs, for example through the plunger driver 203, with one or more linkages 206. Movement of the lid 202 in the opening or closing direction compresses or tensions the drive springs through the linkages 206 to prime the drive springs. For example, the linkages may be slidably coupled to the lid at one end and to the plunge driver at the other. As the lid moves, the linkages 206 slide in a channel in the lid 202 thereby converting the movement of the lid 202 as it hinges closed into a linear movement in the drive spring thereby compressing it or tensioning it depending on if the drive spring is a compression or a tension spring.

A forward facing surface 203a of the plunger driver 203 comprises one or more electrically conductive contact pads 203b in electrical connection with an integrated circuit (not shown) of the auto-injector. The integrated circuit of the auto-injector may comprise a processor in communication with one or more memory devices including secondary storage, read only memory (ROM), and random access memory (RAM). The processor may be implemented as one or more central processor unit chips. The integrated circuit may further comprise input/output (I/O) devices and network connectivity devices, such as a Bluetooth™ module comprising a transceiver configured to establish a connection with an external device such as a smartphone or other user device. The integrated circuit may be powered by a battery. The components of the integrated circuit may be provided on a printed circuit board (PCB) (not shown) of the auto-injector 200. The PCB may be incorporated into plunger driver 203 or into the housing 201 of the auto-injector 200. During firing of the auto-injector, the contact pads 203b on the plunger driver 203 move relative to the housing of the auto-injector. Accordingly, where the PCB of the auto-injector is incorporated into the housing, the contact pads 203b on the plunger driver 203 may be electrically connected to the integrated circuit on the PCB using one or more wires in order to maintain an electrical connection with the EEPROM 111 during firing. The contact pads 203b may be provided on part of the plunger driver 203 or other part of the auto-injector that does not move during firing. In this case, the information stored on the EEPROM 111 is read once on insertion of the safety syringe 100 into the auto-injector 200 and electrical contact may subsequently be broken during firing of the auto-injector 200.

FIG. 2b shows a view of the auto-injector of FIG. 2a with a safety syringe 100 such as that described in connection with FIGS. 1a-1c received therein. The one or more electrically conductive contact pads on the forward facing surface 203a of the plunger driver 203 are arranged to contact the one or more electrically conductive contact pads 110 on the rearward facing surface of the head portion 107 of the safety plunger 106 when the safety syringe 100 is received in the auto-injector. Thus, an electrical connection is established between the input and/or output pins of the EEPROM 111 and the integrated circuit of the auto-injector. The processor is configured to execute instructions, codes, computer programs, scripts and the like which it accesses from the one or more memory devices to read the information stored on the EEPROM 111 and cause the integrated circuit to export the information through the output device and/or network connectivity devices. The exported information may be displayed and/or analysed by an external device such as a smartphone connected to the integrated circuit through the network connectivity devices. The smartphone may give an indication to the user whether or not the correct syringe and/or medicament has been selected for that user based on the information stored in the EEPROM 111. Optionally, the auto-injector may be provided with a visual and/or audible indicator for example one or more light emitting diodes and/or speakers controlled by the integrated circuit and/or external device such as a smartphone and be configured to indicate whether or not a connection has been established with the external device and/or the correct syringe and/or medicament has been selected for the user.

It will be appreciated that the electrical power requirements of reading an EEPROM by establishing an electrical connection to it is substantially less than the electrical power requirements of interrogating an RFID tag with an RFID reader, or operating a camera to scan a QR code. Thus, by using an EEPROM instead of an RFID tag or QR code to store information related to the syringe, the electrical power requirements of the integrated circuit of the auto-injector are reduced and the number of EEPROM readings that the auto-injector can perform before the battery needs to be replaced is greatly increased.

The skilled person will be able to envisage other syringes, auto-injectors and features thereof without departing from the scope of the appended claims. In particular, it is noted that one or more features included in one or more drawings may be integrated into auto-injectors shown in other drawings, as will be appreciated by the skilled person.

For example, the plunger driver may comprise one or more drive springs that may be compression, tension, and or torsion springs. These may be arranged rearwards and aligned in the same axis as the syringe received in the auto-injector. Alternatively, the one or more drive springs may be arranged under, above or adjacent to a plane of the syringe thereby allowing the total length of the device to be reduced compared to devices where the one or more drive springs are arranged rearwards in the same axis as the syringe.

Further, whilst the above exemplary syringe of the drawings is a safety syringe, it is envisaged an EEPROM may also be provided on a standard syringe without a safety plunger. In which case the electrically conductive contact pads connected to the EEPROM input and/or output pins may be provided on the syringe plunger instead of the safety plunger.

1. A syringe comprising a barrel and a needle at an end thereof, a syringe plunger configured to move to cause a medicament within the barrel to be expelled from the needle; and an electrically erasable programmable read-only memory (EEPROM) configured to store information related to the syringe; and
a printed circuit board comprising one or more electrically conductive contact pads electrically connected to one or more input or output pins of the EEPROM wherein the printed circuit board is arranged on a rearward facing surface of the safety plunger.

2. (canceled)

3. The syringe according to claim 1, comprising:
a safety plunger coupled to the plunger such that an inward stroke of the safety plunger causes the syringe plunger to move within the barrel;
a sheath configured to cover the needle after use of the syringe;
wherein the safety plunger is configured to couple to the sheath at a first point on the inward stroke, and is configured to decouple from the syringe plunger at a second point on the inward stroke such that the safety plunger is moveable independently of the syringe plunger,
and wherein further movement of the safety plunger after the first and second points on the inward stroke causes the sheath to cover the needle.

4. (canceled)

5. The syringe according to claim 1, wherein the printed circuit board is arranged on a rearward facing surface of the syringe-plunger.

6. An auto-injector for receiving and operating a syringe to facilitate the injection of a medicament into a subject, the autoinjector comprising:
a plunger driver configured on activation of the auto-injector to drive a plunger of the syringe received in the auto-injector forward within the auto-injector to operate the syringe; and
one or more electrically conductive contact pads configured to contact one or more electrically conductive contact pads on the syringe to establish an electrical connection between an integrated circuit of the auto-injector and an electrically erasable programmable read-only memory (EEPROM) of the syringe.

7. The auto-injector according to claim 6, wherein the one or more electrically conductive contact pads are arranged on a forward facing surface of the plunger driver.

8. The auto-injector according to claim 6, wherein the integrated circuit is configured to read the EEPROM to obtain information relating to the syringe stored on the EEPROM, and export the information to an external device.

9. The auto-injector according to claim 6, wherein the integrated circuit of the auto-injector is configured to communicate with the EEPROM of the syringe using an I2C bus.
